(19) United States
(12) Patent Application Publication
(10) Pub. No.: US 2006/0253109 A1
(43) Pub. Date: Nov. 9, 2006

(54) Surgical robotic helping hand system

(76) Inventor: David Chu, San Gabriel, CA (US)

Correspondence Address: CHAN LAW GROUP LC, 1055 W. 7TH ST,, SUITE 1880, LOS ANGELES, CA, 90017, US

(21) Appl. No.: 11/350,223

(22) Filed: Feb. 8, 2006

(51) Int. Cl. A61B 17/00

(52) U.S. Cl. 606/1

(57) ABSTRACT

A surgical robotic helping hand system according to the present invention enables a surgeon to effectively operate within a patient's body cavities via surgical instruments manually manipulated outside such cavities by holding the various surgical instruments otherwise held by assistant surgeons during the minimally invasive surgical procedure. At least one of the robot-like surgical instrument holders may be specially configured to contain a laparoscopic instrument such that the laparoscope is contained within a sleeve shifter device and capable of being remote controlled for advancement or retraction of the laparoscope along an axis thereof. The robotic-like arms of the surgical instrument holders and laparoscopic instrument holder are attached to a surgical fixation device having a retractor panel that may be adapted to fit a standard Bookwalter retractor device.

### BACKGROUND OF THE INVENTION

1. Field of the Invention

The present invention relates to the field of medical devices, and more specifically to a surgical robotic helping hand system for use in minimally invasive surgical procedures.

2. Description of the Prior Art

Minimally invasive surgical procedures typically employ small incisions in body cavities for access of various surgical instruments, including forceps, laparoscopes, scalpels, scissors, and the like. It is often the case that several surgical hands, such as several laparoscopic instrument and camera holders, are necessary to hold these instruments for the operating surgeon during the particular surgical procedure.

With the introduction of robotic-assisted minimally invasive surgery (MIS) in recent years, hospitals worldwide have made significant investments in acquiring this latest technology for their respective facilities. A number of robotic systems have been specifically developed for MIS and a few of these robotic systems are commercially available on the market, such as the da Vinci Surgical System (Intuitive Surgical), ZEUS Robotic Surgical System (Computer Motion), and AESOP Robotic System (Computer Motion). Present surgical instrument holders of these commercial robotic systems, such as those for laparoscopes, are concerned with complex movements and degrees of movement that can be remote controlled. These commercial robotic systems may cost hospitals as much as $1.5 million to acquire and may require over $100,000 in maintenance expenses each year. In addition, there is a steep learning curve in using this technology such that costly special training and experience are necessary in order to achieve optimum results. This may require additional specially trained personnel to manage the technical aspects of the surgical procedures. Commercial robotic systems also require a significant amount of space in the operating room and an increased time to set up the equipment. Further, the use of such highly sophisticated technology typically increases the cost of the minimally invasive surgical procedure in the thousands of dollars.

It would be desirable to have a surgical robotic helping hand system that replaces the hands of assisting surgeons in the operating room and the use of complicated and expensive robotic systems currently on the market. It would be also desirable to have a surgical robotic helping hand system that is low cost and allows minimally invasive surgical procedures to be conducted on the same scale as hospitals using the more complicated and higher cost commercially available robotic systems. It would be further desirable to have a surgical robotic helping hand system that has manually set robotic arms, which can be easily disassembled, cleaned and sterilized, and has a short set up time. Finally, it would be desirable to have a surgical robotic helping hand system that has simpler, slender, and easier to operate and maneuver robotic arms securing the latest in laparoscopic instruments with a motor driven one-dimensional movement controlled by a foot pedal or hand-operated lever.

### SUMMARY OF THE INVENTION

The present invention provides a surgical robotic helping hand system for use in minimally invasive surgical procedures. In a first aspect, the present invention includes a surgical fixation device having an elongate support bar, and extension bar, and a retractor panel. The lower portion of the elongate support bar includes locking means for vertically attaching to a side bar of an operating table, and the upper portion of the elongate support bar includes locking means for adjustably connecting to a proximal end of the extension bar at a desired height. The distal end of the extension bar includes locking means for adjustably connecting to the retractor panel in which the retractor panel is positioned substantially over a patient on the operating table. The retractor panel may assume a shape in the form of a semi-circular bar, a U-shaped bar, or a substantially rectangular member having an open interior portion. The retractor panel may include at least one retractor blade.

A plurality of brackets is slidably connected to the retractor panel and has adjustable clamp means by way of clasps for attaching to the retractor panel. A plurality of surgical instrument holders includes means for firmly grasping surgical instruments by way of a vise like clamp. An elongate cup portion is affixed to the bracket so that a lower base portion of the surgical instrument holder may be positioned into the elongate cup portion. At least one elongate cup portion may be parallel the patient's abdominal surface. Similarly, at least one elongate cup portion may be perpendicular to the patient's abdominal surface.

The surgical instrument holder further includes an extender portion and a grasping member with an open interior portion at a distal end thereof to secure the surgical instrument. The surgical robotic helping hand system may accommodate eight surgical instruments of various types. The extender portion is affixed to the outside surface of the grasping member and may be removable and elastic, flexible, or non-flexible. The extender portion can be set into a fixed position by a lever, switch, or rotating knob device. At the intersection of the extender portion and grasping member there is an adjustable swivel in which the angle of the surgical instrument holder with respect to the retractor panel may be changed.

The grasping member includes releasing means by way of a lever for adjusting the surgical instrument on a level and rotation of the grasp. The grasping member may accommodate suctioning devices, operating devices carrying electrical power, light sources, or lasers. The surgical robotic helping hand system further includes a laparoscopic instrument holder configured to contain a laparoscopic instrument such that the laparoscope is contained within a sleeve shifter device.

In another aspect of the present invention, the surgical robotic helping hand system includes a surgical fixation device having an elongate support bar, and extension bar, and a retractor panel. The lower portion of the elongate support bar includes locking means for vertically attaching to a side bar of an operating table, and the upper portion of the elongate support bar includes locking means for adjustably connecting to a proximal end of the extension bar at a desired height. The distal end of the extension bar includes locking means for adjustably connecting to the retractor panel in which the retractor panel is positioned substantially over a patient on the operating table.

A plurality of brackets is slidably connected to the retractor panel and has adjustable clamp means for attaching to the retractor panel. A plurality of surgical instrument holders includes means for firmly grasping surgical instruments. An elongate cup portion is affixed to the bracket so that the bottom portion of the surgical instrument holder may be positioned into the elongate cup portion.

The surgical instrument holder further includes an extender portion and a grasping member with an open interior portion at a distal end thereof to secure the surgical instrument. The extender portion is affixed to the outside surface of the grasping member.

The grasping member includes releasing means for adjusting the surgical instrument on a level and rotation of the grasp. At least one of the surgical instrument holders may be specially configured to contain a laparoscopic instrument such that the laparoscope is contained within a sleeve shifter device and is capable of being remote controlled for advancement or retraction of the laparoscope along an axis of the sleeve shifter device.

In a further aspect of the present invention, the surgical robotic helping hand system includes a surgical fixation device having an elongate support bar, and extension bar, and a retractor panel. The lower portion of the elongate support bar includes locking means for vertically attaching to a side bar of an operating table, and the upper portion of the elongate support bar includes locking means for adjustably connecting to a proximal end of the extension bar at a desired height. The distal end of the extension bar includes locking means for adjustably connecting to the retractor panel in which the retractor panel is positioned substantially over a patient on the operating table.

A plurality of brackets is slidably connected to the retractor panel and has adjustable clamp means for attaching to the retractor panel. A plurality of surgical instrument holders includes means for firmly grasping surgical instruments. An elongate cup portion is affixed to the bracket so that a lower base portion of the surgical instrument holder may be positioned into the elongate cup portion.

The surgical instrument holder further includes an extender portion and a grasping member with an open interior portion at a distal end thereof to secure the surgical instrument. The extender portion is affixed to the outside surface of the grasping member. The grasping member includes a lever for adjusting the surgical instrument on a level and rotation of the grasp. The lever in a first position converts the extender portion into a stiff, immobile, curvaceous rod in the shape set in, and the lever in a second position sets the grasping member in a closed position while holding the surgical instrument. The extender portion is capable of being moved in a multitude of orientations prior to being set into a fixed position.

The surgical robotic helping hand system further includes a laparoscopic instrument configured to contain a laparoscopic instrument such that the laparoscope is contained within a sleeve shifter device and is capable of being advanced or and retracted along an axis of the sleeve shifter device.

These and other features and advantages of this invention will become further apparent from the detailed description and accompanying figures that follow. In the figures and description, numerals indicate the various features of the disclosure, like numerals referring to like features throughout both the drawings and the description.

### BRIEF DESCRIPTION OF THE DRAWINGS

FIG. 1 is a perspective view of the surgical robotic helping hand system according to the present invention attached to a side bar of an operating table.

FIG. 2A is an enlarged perspective view of an elongate cup portion affixed to a bracket and configured such that the elongate cup portion is parallel to a patient's abdominal surface in one embodiment of the present invention.

FIG. 2B is an enlarged perspective view of an elongate cup portion affixed to a bracket and configured such that the elongate cup portion is perpendicular to a patient's abdominal surface in a further embodiment of the present invention.

FIG. 3 is an enlarged perspective view of a surgical instrument holder according to the present invention.

FIG. 4A is an enlarged perspective view of a grasping member and distal end of an extender portion of the surgical instrument holder according to the present invention.

FIG. 4B is an enlarged perspective view of the grasping member and distal end of the extender portion of FIG. 4A when a lever is depressed in a first position according to the present invention.

FIG. 4C is an enlarged perspective view of the grasping member and distal end of the extender portion of FIG. 4A when a lever is depressed in a second position according to the present invention.

FIG. 5A is an enlarged perspective view of a laparoscopic instrument holder configured to hold a laparoscopic instrument according to the present invention.

FIG. 5B is a perspective view of the laparoscopic instrument holder of FIG. 5A containing a laparoscope according to the present invention.

FIG. 5C is an enlarged perspective view of a sleeve shifter device of the laparoscopic instrument holder of FIG. 5A shown in an open position according to the present invention.

### DETAILED DESCRIPTION OF THE PREFERRED EMBODIMENTS

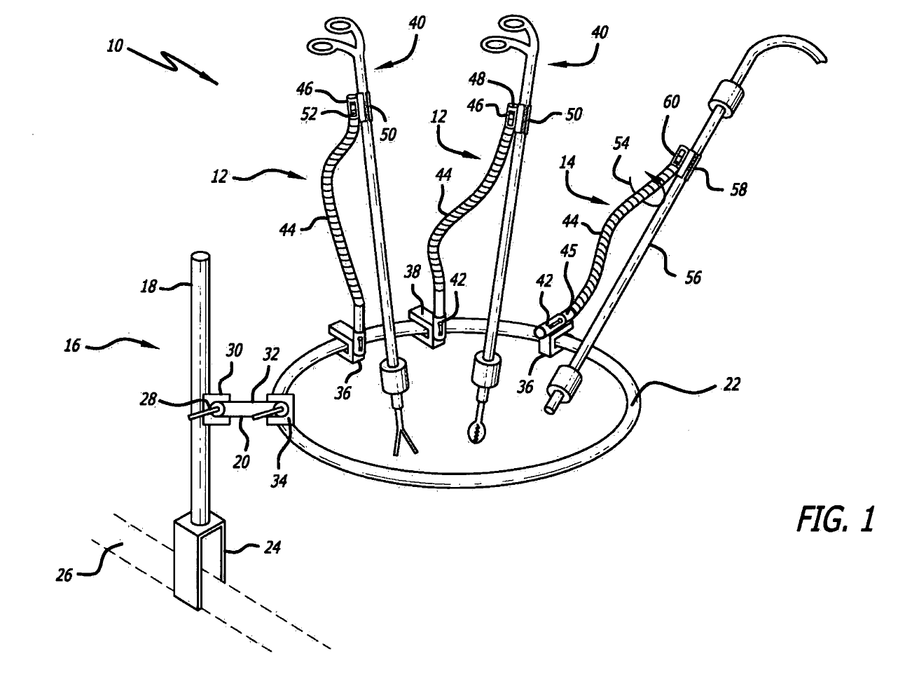

The surgical robotic helping hand system 10 of the present invention is generally illustrated in FIG. 1. The robotic-like arms of the surgical instrument holders 12 and laparoscopic instrument holder 14 are attached to a surgical fixation device 16. The surgical fixation device 16 of the surgical robotic helping hand system 10 includes an elongate support bar 18, an extension bar 20, and a retractor panel 22. The retractor panel 22 may be adapted to fit onto a Bookwalter retractor device, which is present in most hospital operating rooms. A suitable retractor device of the Bookwalter type is described in U.S. Pat. No. 4,254,763.

At the lower portion of the elongate support member 18 is a first adjustable clamp assembly 24 that secures the elongate support bar 18 to a surgical operating room table 26. Following proper tightening and positioning of the adjustable clamp assembly 24, the elongate support member 18 extends upward in a vertical direction as shown in FIG. 1. At the upper portion of the elongate support member 18 is a second adjustable clamp assembly 28 that secures a proximal end 30 of the extension bar 20 thereto at a desired height. At a distal end 32 of the extension bar 20 is a third adjustable clamp assembly 34 that connects to the retractor panel 22. The retractor panel 22 is positioned substantially over a patient on the surgical operating room table 26. In various embodiments of the present invention, the retractor panel 22 may assume various shapes, including a semi-circular bar (not shown), a U-shaped bar (not shown), and a substantially rectangular member having an open interior portion as shown in FIG. 1. At least one retractor blade (not shown) may be optionally adjustably mounted on the retractor panel 22.

As shown in FIG. 1, a plurality of brackets 36 may be slidably positioned onto the retractor panel 22. Each of the brackets 36 are configured to have an adjustable clamp assembly or clasp 38 in order for the brackets 36 to attach to the retractor panel 22.

The surgical robotic helping hand system 10 further includes a plurality of surgical instrument holders 12 configured to firmly grasp various types of surgical instruments 40, including, but not limited to, forceps, scalpels, or scissors, as shown in FIG. 1. It is contemplated by the present invention that the surgical robotic helping hand system 10 may effectively accommodate up to eight surgical instruments 40 during a typical minimally invasive surgery (MIS) procedure. An elongate cup portion 42 is directly affixed to each bracket 36 by soldering or welding such that a lower base portion 45 of the surgical instrument holder 12 may be securely positioned into the elongate cup portion 42 as shown in FIG. 1.

Referring further to FIG. 1, the surgical instrument holder 12 includes an extender portion 44 and a grasping member 46 at a distal end 48 of the surgical instrument holder 12. The extender portion 44 is directly affixed to an outside surface of the grasping member. The grasping member 46 firmly grasps surgical instruments 40 using a vise-like clamp 50 in one embodiment. The present invention contemplates that the grasping member 46 may releasably adjust the surgical instrument 40 contained within the surgical instrument holder 12 by a lever 52 on a level and rotation of the grasp. The extender portion 44 is capable of being manipulated in a multitude of orientations such as that indicated by arrows 54 prior to being set into a fixed position.

In one embodiment of the invention, extender portion 44 may be fabricated from a metal alloy that has a flexible helical wrap such as a goose neck lamp extender and a central flexible rod. When the rod of the extender portion 44 is firmly pulled against the helical wrap, the self-fitting helix becomes solid as the rod and helical wrap fit securely against each other.

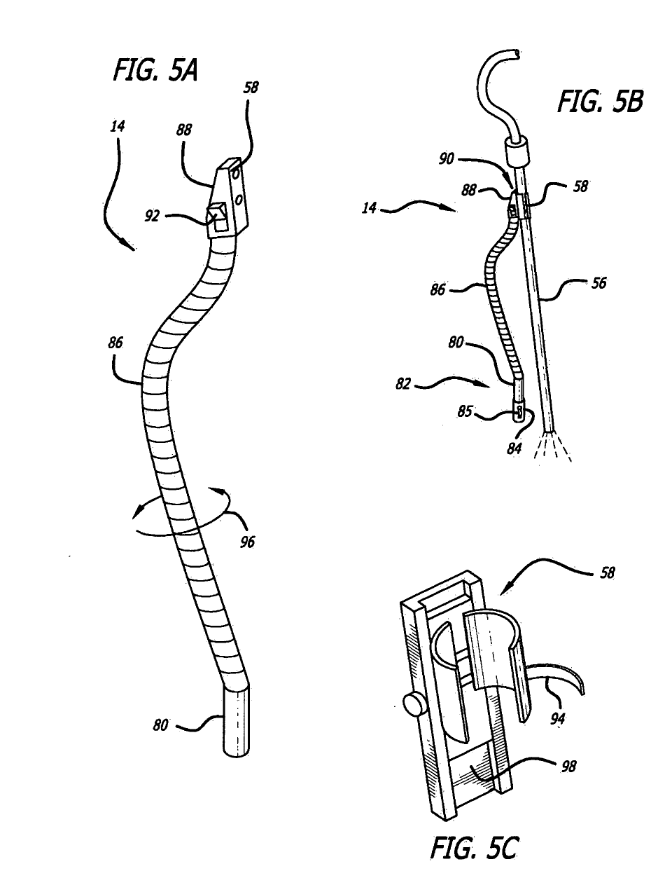

The surgical robotic helping hand system 10 further includes a laparoscopic instrument holder 14 configured to contain a laparoscopic instrument 56. The laparoscopic instrument 56 may be manually positioned inside a sleeve shifter device 58 of the laparoscopic instrument holder 14. In one embodiment, the sleeve shifter device 58 has the capability to advance and retract the laparoscopic instrument 56 along an axial shaft (shown in FIG. 5C) of the sleeve shifter device 58 using a hand-operated lever or switch 60. A further embodiment provides that the sleeve shifter device 58 has the capability to advance and retract the laparoscopic instrument 56 along an axial shaft (shown in FIG. 5C) of the sleeve shifter device 58 using a remote control or foot pedal. The motor that advances the sleeve shifter device 58 forward and backward is battery operated and can be switched on and off, and activated for forward or reverse movement by a hand or foot operated switch connected by chord or by remote control.

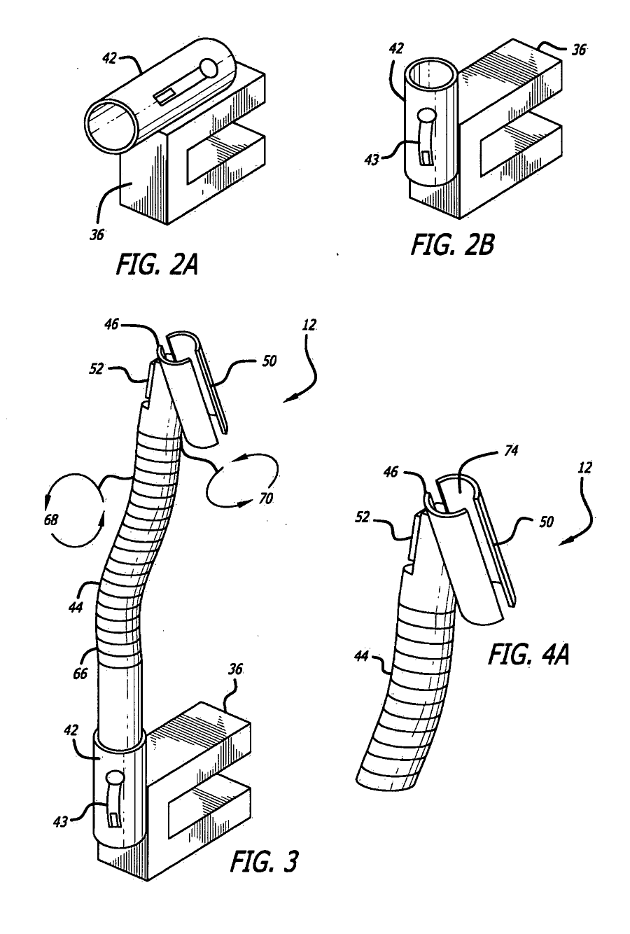

Referring now to FIG. 2A, the elongate cup portion 42 affixed to the bracket 36 is shown in more detail. In one embodiment, elongate cup portion 42 is configured to be parallel 62 to a patient's abdominal surface (not shown) when mounted on the retractor panel 22. In a further embodiment shown in FIG. 2B, the elongate cup portion 42 affixed to the bracket 36 is configured to be perpendicular 64 to the patient's abdominal surface (not shown) when mounted on the retractor panel 22. These different configurations of the elongate cup portion 42 enable the surgical instrument holder 12 to accommodate various types of surgical instruments 40 during the MIS procedure.

FIG. 3 illustrates the surgical instrument holder 12 in more detail. Lower base portion 45 of the surgical instrument holder 12 is securely positioned into the elongate cup portion 42. Proximal end 66 of the extender portion 44 is directly affixed to lower base portion 45. Elongate cup portion 42 is configured to have a lever or switch 43 mounted onto an outside surface of the elongate cup portion 42. The lever 43 secures the lower base portion 45 after being positioned into the elongate cup portion 42.

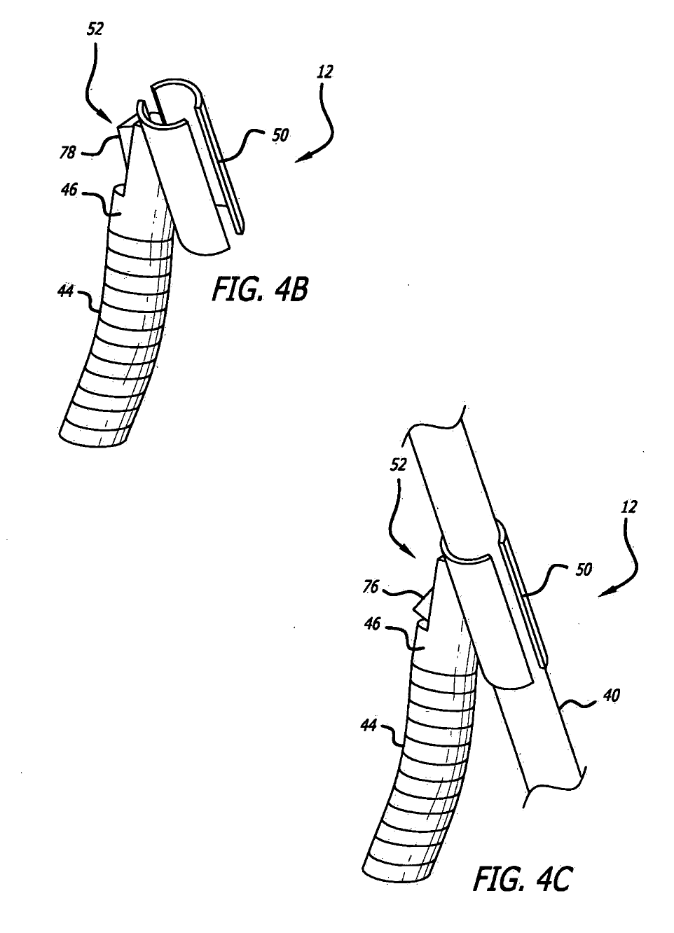

The surgical instrument holder 12 of the surgical robotic helping hand system 10 provides the surgeon with several degrees of freedom. In one embodiment, the extender portion 44 of the surgical instrument holder 12 may be elastic or flexible and manipulated by the surgeon into various positions as shown by arrow 68. In additional embodiments, the extender portion 44 may be converted into a non-flexible position as required by the particular MIS procedure. The extender portion 44 may be set into a fixed position using a lever or switch 52 (shown in FIGS. 4B-C) or rotating knob device mounted on the outside surface of the grasping member 46. Further, at an intersection of the extender portion 44 and the grasping member 46 there is an adjustable swivel as shown by arrow 70 in which the angle of the surgical instrument holder 12 with respect to the retractor panel 22 may be adjusted.

Referring now to FIG. 4A, the grasping member 46 and a distal end 72 of the extender portion 44 of the surgical instrument holder 12 are shown in more detail. The grasping member 46 further includes an open interior portion 74 capable of securing the surgical instrument 40 when grasping member 46 is in a closed position. Grasping member 46 firmly grasps surgical instruments 40 using vise-like clamp 50. Lever or switch 52 for setting the position of extender portion 44 and adjusting grasp of vise-like clamp 50 is shown mounted on the outside surface of the grasping member 46. Grasping member 46 of the surgical instrument holder 12 is configured to accommodate various types of devices, such as suctioning devices, operating devices carrying electrical power, light-sources, or lasers, required for the particular MIS procedure conducted on the patient.

FIG. 4B illustrates the grasping member 46 and the distal end 72 of the extender portion 44 of FIG. 4A when lever 52 is depressed in a first position 78 in one embodiment of the present invention. In first position 78, the flexible extender portion 44 is converted into a stiff, immobile, curvaceous rod in the shape set in.

FIG. 4C illustrates the grasping member 46 and the distal end 72 of the extender portion 44 of FIG. 4A when lever 52 is depressed in a second position 76 in a further embodiment of the present invention. In second position 76, vise-like clamp 50 of grasping member 46 is set in a closed position while holding surgical instrument 40.

Referring now to FIG. 5A, the laparoscopic instrument holder 14 configured to hold a laparoscopic instrument 56 in accordance with the present invention is shown in more detail. Specifically, the laparoscopic instrument holder 14 is shown without the sleeve shifter device 58 of FIG. 5C docked onto the distal end 90 of upper base portion 88.

FIG. 5B illustrates the laparoscopic instrument holder 14 of FIG. 5A containing a laparoscopic instrument 56. The laparoscopic instrument holder 14 further includes a lower base portion 80 at a proximal end 82 of laparoscopic instrument holder 14. Lower base portion 80 is positioned into elongate cup portion 84. Extender portion 86 is affixed to an outside surface of an upper base portion 88 at a distal end 90 of laparoscopic instrument holder 14. Elongate cup portion 84 is configured to have a lever or switch 85 mounted onto an outside surface of the elongate cup portion 84. The lever 85 secures the lower base portion 80 after being positioned into the elongate cup portion 84.

Similar to the extender portion 44 of the surgical instrument holder 12, extender portion 86 of the laparoscopic instrument holder 14 may be fabricated from a metal alloy that has a flexible helical wrap, such as a goose neck lamp extender and a central flexible rod. When the rod of the extender portion 86 is firmly pulled against the helical wrap, the self-fitting helix becomes solid as the rod and helical wrap fit securely against each other.

Referring further to FIG. 5B, sleeve shifter device 58 is affixed to upper base portion 88 at the distal end 90 of laparoscopic instrument holder 14. The sleeve shifter device 58 is positioned flush to the upper base portion 88 in a vertical direction.

The laparoscopic instrument holder 14 of the surgical robotic helping hand system 10 provides the surgeon with several degrees of freedom similar to that indicated above for the surgical instrument holder 12. In one embodiment (shown in FIGS. 5A-C), a lever or switch 92 mounted on an outside surface of the upper base portion 88 sets the desired position of flexible extender portion 86 and operates clasp 94 of the sleeve shifter device 58 into an open or close position for securing the laparoscopic instrument 56. The extender portion 86 is capable of being manipulated in a multitude of orientations such as that indicated by arrow 96 prior to being set into a fixed position.

FIG. 5C illustrates the sleeve shifter device 58 of the laparoscopic instrument holder 14 of FIG. 5A with clasp 94 in an open position. The sleeve shifter device 58 advances and retracts the laparoscopic instrument 56 along axial shaft 98 of sleeve shifter device as desired by the surgeon during the MIS procedure.

It is contemplated by the present invention that each of the surgical instrument holders 12 and laparoscopic instrument holders 14 used in accordance with the surgical robotic helping hand system 10 can withstand high temperatures during the sterilization process, and thus can be used again in subsequent MIS procedures after proper sterilization.

Having now described the invention in accordance with the requirements of the patent statutes, those skilled in the art will understand how to make changes and modifications in the present invention to meet their specific requirements or conditions. Such changes and modifications may be made without departing from the scope and spirit of the invention as set forth in the following claims.

What is claimed is:

1. A surgical robotic helping hand system for use in minimally invasive surgical procedures, comprising:
a surgical fixation device having an elongate support bar, an extension bar, and a retractor panel;
a lower portion of the elongate support bar including locking means for vertically attaching to a side bar of an operating table, and an upper portion of the elongate support bar including locking means for adjustably connecting to a proximal end of the extension bar at a desired height;
a distal end of the extension bar including locking means for adjustably connecting to the retractor panel, wherein the retractor panel is positioned substantially over a patient on the operating table;
a plurality of brackets slidably connected to the retractor panel and having adjustable clamp means for attaching to the retractor panel;
a plurality of surgical instrument holders including means for firmly grasping surgical instruments;
an elongate cup portion affixed to the bracket, wherein a lower base portion of the surgical instrument holder is positioned into the elongate cup portion;
the surgical instrument holder further including an extender portion and a grasping member at a distal end thereof, wherein the extender portion is affixed to an outside surface of the grasping member;
the grasping member including releasing means for adjusting the surgical instrument on a level and rotation of the grasp;
the extender portion capable of being moved in a multitude of orientations prior to being set into a fixed position; and
a laparoscopic instrument holder configured to contain a laparoscopic instrument such that the laparoscope is contained within a sleeve shifter device.

2. The system of claim 1, wherein the retractor panel is a semi-circular bar.

3. The system of claim 1, wherein the retractor panel is a U-shaped bar.

4. The system of claim 1, wherein the retractor panel is a substantially rectangular member having an open interior portion.

5. The system of claim 1, wherein the retractor panel includes at least one retractor blade.

6. The system of claim 1, wherein the system may accommodate eight surgical instruments.

7. The system of claim 1, wherein the adjustable clamp means include clasps being capable of attachment to the retractor panel.

8. The system of claim 1, wherein the means for firmly grasping surgical instruments include a vise like clamp.

9. The system of claim 1, wherein the releasing means for adjusting the surgical instrument on a level and rotation of the grasp includes a lever.

10. The system of claim 9, wherein the lever in a first position converts the extender portion into a stiff, immobile, curvaceous rod in the shape set in, and the lever in a second position sets the grasping member in a closed position while holding the surgical instrument.

11. The system of claim 1, wherein the extender portions are removable.

12. The system of claim 1, wherein the extender portions are elastic.

13. The system of claim 1, wherein the extender portions are flexible.

14. The system of claim 1, wherein the extender portions are non-flexible.

15. The system of claim 1, wherein at least one elongate cup portion affixed to the bracket is parallel to an abdominal surface of the patient.

16. The system of claim 1, wherein at least one elongate cup portion affixed to the bracket is perpendicular to an abdominal surface of the patient.

17. The system of claim 1, wherein at least one elongate cup portion affixed to the bracket is parallel to an abdominal surface of the patient, and at least one elongate cup portion affixed to the bracket is perpendicular to the abdominal surface of the patient.

18. The system of claim 1, the grasping member further including:
an open interior portion capable of securing the surgical instrument when grasping member is in a closed position.

19. The system of claim 1, wherein the surgical instrument holder accommodates forceps, scalpels, or scissors.

20. The system of claim 1, wherein the grasping member of the surgical instrument holder accommodates suctioning devices, operating devices carrying electrical power, light-sources, or lasers.

21. The system of claim 1, wherein the extender portion is set into a fixed position by at least one of a lever, switch, and rotating knob device.

22. The system of claim 1, wherein at an intersection of the extender portion and grasping member there is an adjustable swivel in which the angle of the surgical instrument holder with respect to the retractor panel may be changed.

23. The system of claim 1, wherein the sleeve shifter device advances and retracts the laparoscopic instrument along an axial shaft of the sleeve shifter device by a foot control pedal.

24. The system of claim 1, wherein the sleeve shifter device advances and retracts the laparoscopic instrument along an axial shaft of the sleeve shifter device by a hand-operated lever.

25. The system of claim 1, wherein the laparoscope is capable of being remote controlled for advancement or retraction of the laparoscope along an axis of the sleeve shifter device.

26. The system of claim 1, the laparoscopic instrument holder further including:
a lower base portion at a proximal end of the laparoscopic instrument holder positioned into the elongate cup portion; and
an extender portion affixed to an outside surface of an upper base portion at a distal end of the laparoscopic instrument holder.

27. The system of claim 26, wherein the sleeve shifter device is mounted on the upper base portion at the distal end of the laparoscopic instrument holder.

28. A surgical robotic helping hand system for use in minimally invasive surgical procedures, comprising:
a surgical fixation device having an elongate support bar, an extension bar, and a retractor panel;
a lower portion of the elongate support bar including locking means for vertically attaching to a side bar of an operating table, and an upper portion of the elongate support bar including locking means for adjustably connecting to a proximal end of the extension bar at a desired height;
a distal end of the extension bar including locking means for adjustably connecting to the retractor panel, wherein the retractor panel is positioned substantially over a patient on the operating table;
a plurality of brackets slidably connected to the retractor panel and having adjustable clamp means for attaching to the retractor panel;
a plurality of surgical instrument holders including means for firmly grasping surgical instruments;
an elongate cup portion affixed to the bracket, wherein a lower portion of the surgical instrument holder is positioned into the elongate cup portion;
the surgical instrument holder further including an extender portion and a grasping member having an open interior portion at a distal end of the surgical instrument holder to secure the surgical instrument, wherein the extender portion is affixed to an outside surface of the grasping member;
the grasping member including releasing means for adjusting the surgical instrument on a level and rotation of the grasp;
the extender portion capable of being moved in a multitude of orientations prior to being set into a fixed position; and
a laparoscopic instrument holder configured to contain a laparoscopic instrument such that the laparoscope is contained within a sleeve shifter device and capable of being remote controlled for advancement or retraction of the laparoscope along an axis of the sleeve shifter device.

29. A surgical robotic helping hand system for use in minimally invasive surgical procedures, comprising:
a surgical fixation device having an elongate support bar, an extension bar, and a retractor panel;
a lower portion of the elongate support bar including locking means for vertically attaching to a side bar of an operating table, and an upper portion of the elongate support bar including locking means for adjustably connecting to a proximal end of the extension bar at a desired height;
a distal end of the extension bar including locking means for adjustably connecting to the retractor panel, wherein the retractor panel is positioned substantially over a patient on the operating table;
a plurality of brackets slidably connected to the retractor panel and having adjustable clamp means for attaching to the retractor panel;
a plurality of surgical instrument holders including means for firmly grasping surgical instruments;
an elongate cup portion affixed to the bracket, wherein a lower portion of the surgical instrument holder is positioned into the elongate cup portion;
the surgical instrument holder further including an extender portion and a grasping member having an open interior portion at a distal end thereof to secure the surgical instrument, wherein the extender portion is affixed to an outside surface of the grasping member;
the grasping member including a lever for adjusting the surgical instrument on a level and rotation of the grasp, wherein the lever in a first position converts the extender portion into a stiff, immobile, curvaceous rod in the shape set in, and the lever in a second position sets the grasping member in a closed position while holding the surgical instrument;
wherein the extender portion is capable of being moved in a multitude of orientations prior to being set into a fixed position; and
a laparoscopic instrument holder configured to contain a laparoscopic instrument such that the laparoscope is contained within a sleeve shifter device, wherein the laparoscope is capable of being advanced and retracted along an axis of the sleeve shifter device.
